(19) United States
(12) Patent Application Publication
(10) Pub. No.: US 2019/0268346 A1
(43) Pub. Date: Aug. 29, 2019

(54) LIFE DOCUMENT SECURING SYSTEM AND METHOD FOR AT-RISK YOUTH

(71) Applicant: Living Advantage, Inc., Hollywood, CA (US)

(72) Inventors: Pamela R. Clay, Hollywood, CA (US); Eugenia Wilson, Hollywood, CA (US); JaniceElizabeth Kreh, Hollywood, CA (US)

(73) Assignee: Living Advantage, Inc., Hollywood, CA (US)

(21) Appl. No.: 16/289,155

(22) Filed: Feb. 28, 2019

Related U.S. Application Data
(63) Continuation of application No. 14/945,236, filed on Nov. 18, 2015, now abandoned.

(51) Int. Cl. H04L 29/06; G16H 10/60

(52) U.S. Cl. CPC H04L 63/104; H04L 63/083; G16H 10/60

(57) ABSTRACT

An at-risk youth protection system is disclosed for securing and enabling authorized access to one or more life documents of youth. The system comprising a server including a database that stores life documents in accounts associated with a plurality of youth users of the system. The database further includes a user management system that controls authorization and security levels for system administrator users, case manager users, and youth users. The system administrator users have full access and editing privileges to all the accounts associated with the plurality of youth users. The case manager users have full access and editing privileges to only accounts associated with youth users to which they have been assigned. The youth users only have access privileges to their own accounts.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD

This description generally relates to a computer-based, web-based, life document securing system and method for “at-risk” youth.

### BACKGROUND

Local statistics on the at-risk “system-involved” youth (i.e., youth who are connected to the child welfare system, juvenile justice system, public welfare system, foster care system, or combinations thereof) show that about 25,000 youth aged 16 and older transition from the said systems each year. Studies also show these youth often do not get the help they need to complete high school, gain employment, access health care, enroll in postsecondary education, obtain and stable housing and life-sustaining resources and supplies. Consequently, at-risk “system-involved” youth have significantly lower success rates than their non-involved peers in such areas as graduation and full-time employment. Moreover, said youth are at a higher risk for and experience higher incidences of school dropout, delinquency, substance abuse, teen pregnancy and parenting, and homelessness. Without an entity providing constructive activities, consistent support, and continuing education, at-risk “system-involved” youth in have no formal education, engage in risky behaviors, get involved in drugs and gangs, and ultimately end up in jail or on the streets. Furthermore, the pre-existing emotional and behavioral problems from abandonment, abuse, neglect and overall lack of support within the system, only compound the difficulties for any helpful services.

For example, Los Angeles County has the highest population of “system-involved” youth in the country, with its numbers totaling almost 60,000. Annually, approximately 3,000 youth are forced out of the system without support due to their age, regardless of needs or circumstances, a process known as “aging-out.” Overall, California's data indicates that outcomes for youth “aging-out” are deficient in education, employment, income, and housing, all of which are key indicators for self-sufficiency.

Currently, many of these “system-involved” youth lack access to their vital life documents, including but not limited to, social security cards, birth certificates, and picture identification. These documents are commonly lost or simply not transferred as a “system-involved” youth is relocated to as many as 20 different placements. As a result, “system-involved” youth lose their sense of identity and may even forget their date of birth. Most often, youth without identification face tremendous difficulties end up uneducated, unemployed, homeless and incarcerated. Therefore, the “system-involved” youth need support in the areas of academic achievement, college readiness, career exploration, job training, as well as access to health care, stable housing, and life-sustaining resources. Moreover, to learn life skills and to build social support to promote and maintain self-sufficiency.

Many providers in the child welfare industry have recognized that the biggest problem facing these “system-involved” youth is their inability to access their life documents that not only solidifies their identity in society, but also aids in school enrollment, obtaining employment, securing housing, and accessing medical services. Without these important resources and documentation, youth are unidentifiable, and become disenfranchised and unable to succeed. Furthermore, their marginalization further impacts society by reducing the amount of eligible workforce and tax-paying citizens, increasing violence and drug use, and burdening the public welfare, criminal justice, and emergency medical services systems.

Therefore, there is a continuing need in the industry for a secure centralized system and/or method to store the vital life information of the youth in foster care (or other “system-involved” care) which can be accessed and updated electronically from any location by the corresponding youth, their case workers and the administrators of the system.

### SUMMARY

Briefly, and in general terms, an “at-risk youth” protection system is disclosed for securing and enabling authorized access to one or more life documents. The system comprises a server including a database that stores life documents in accounts associated with a plurality of “system-involved” youth users of the system. The database further includes a user management system that controls authorization and security levels for system administrator users, case manager users, and “system-involved” youth users. The system administrator users have full access and editing privileges to all the accounts associated with the plurality of “system-involved” youth users, as well as back-end access to server, code and application infrastructure. The case manager users only have full access and editing privileges to only accounts associated with “system-involved” youth users to which they have been assigned. The “system-involved” youth users only have access privileges to their own accounts.

The system further comprises a server processor in communication with the database. The processor is configured to execute instructions to access or edit accounts of the plurality of “system-involved” youth users depending on the authorization and security level of a requesting user. The editing of an account of a “system-involved” youth user includes uploading a life document to an account, updating information associated with a youth user, or combinations thereof. Additionally, the system including a network that connects the server to end user terminals associated with the system administrator users and case manager users. The network is configured to receive and process requests from “system-involved” youth users. The system administrator users are authorized to change assignments between case manager users and “system-involved” youth users, which immediately alters youth users' accounts to which the case manager users have access and editing privileges, terminating access to prior assigned youth users' accounts, and granting access to newly assigned youth users' accounts.

In an aspect of one embodiment, the life documents include but not limited to, social security cards, birth certificates, driver's license, photo identification, immunization cards, health records, school records, professional certificates/degrees, legal documents, naturalization/immigration documents, court-appointed treatment certifications, or combinations thereof. In another aspect, the system is configured to receive and process requests from youth users using mobile devices. In still another aspect, the system requires case manager authorization in addition to “system-involved” youth user authorization. In yet another aspect, user authorization includes providing a valid user ID and password. In another aspect, the system records whenever access is attempted or granted to create a record of user activity. In yet another aspect users supply a one-time emailed security code to gain authorization.

In an aspect of another embodiment, system enables customization of the accounts of youth with different security settings depending on youth characteristics. In another aspect, the youth characteristics that contribute to the customized security settings include the age and background of the youth. In still another aspect, under a first customized security setting, youth have authorization to view their own life documents from the database. Under a second customized security setting, the system requires authorization by a case manager in addition to a youth providing authorization for the youth to view their own life documents from the database. Under a third customized security setting, the system requires an administrator user or case manager user at an associated end user terminal to provide authorization in addition to a youth providing authorization for the youth to view their own life documents from the database.

In another embodiment, an “at-risk youth” protection system is also disclosed for securing and enabling authorized access to one or more life documents of youth. The system comprises a server including a database that stores life documents in accounts associated with a plurality of youth users of the system. The database further includes a user management system that controls authorization and security levels for system administrator users, case manager users, and youth users. The system administrator users have full access and editing privileges to all the accounts associated with the plurality of youth users. The case manager users have full access and editing privileges to only accounts associated with youth users to which they have been assigned. The “system-involved” youth users only have access privileges to their own accounts.

The system further comprises a server processor in communication with the database. The processor is configured to execute instructions to access or edit accounts of the plurality of “system-involved” youth users depending on the authorization and security level of a requesting user. The editing of an account of a “system-involved” youth user includes uploading a life document to an account, updating information associated with a youth user, or combinations thereof. Additionally, the system includes a network that connects the server to end user terminals associated with the system administrator users and case manager users. The network is configured to receive and process requests from “system-involved” youth users.

The database further includes (1) a partition that is inaccessible by a “system-involved” youth that stores one or more life documents, and (2) a partition that is accessible by a “system-involved” youth that provides valid authentication credentials. In this embodiment, (1) the “system-involved” youth contacts an associated case manager to request access to the youth's stored life documents, (2) the case manager verifies an identity of the requesting, (3) the case manager provides valid authentication credentials to the system, (4) wherein the case manager moves the requesting youth's life documents from the inaccessible partition of the database to the accessible partition of the database for access by the “system-involved” youth after the youth provides valid authentication credentials, and (5) the “system-involved” youth provides valid authentication credentials to the system and then is provided access to his/her life documents.

Features and advantages will become apparent from the following detailed description, taken in conjunction with the accompanying drawings, which illustrate by way of example, the features of the various embodiments.

### BRIEF DESCRIPTION OF THE DRAWINGS

The accompanying drawings, which are included as part of the present specification, illustrate various embodiments and together with the general description given above and the detailed description of the various embodiments given below serve to explain and teach the principles described herein.

FIG. 1 illustrates a relationship diagram between the Life Document Electronic Security System for “at-risk” Youth (LDESSY) and the different personnel (e.g., system administrator, social worker/case manager, youth, and the like) with authorization to access the LDESSY.

FIG. 2 illustrates a diagram of different functions of different personnel, for example, system administrator, social worker/case manager, youth, and the like, who are the users of the LDESSY, based on their authorization level.

The figures are not necessarily drawn to scale and elements of similar structures or functions are generally represented by like reference numerals for illustrative purposes throughout the figures. The figures or text show examples of possible implementations of the computer-based, life document electronic security system and method for “at-risk” youth, however the teachings are applicable to other implementations without deviating from the present disclosure. The figures are only intended to facilitate the description of the various embodiments described herein. The figures do not describe every aspect of the teachings disclosed herein and do not limit the scope of the claims.

### DETAILED DESCRIPTION

Each of the features and teachings disclosed herein can be utilized separately or in conjunction with other features and teachings to provide a system and method of implementing a computer-based, life document securing system and method for “at-risk” youth. Representative examples utilizing many of these additional features and teachings, both separately and in combination, are described in further detail with reference to the attached figures. This detailed description is merely intended to teach a person of skill in the art further details for practicing aspects of the present teachings and is not intended to limit the scope of the claims. Therefore, combinations of features disclosed above in the detailed description may not be necessary to practice the teachings in the broadest sense, and are instead taught merely to describe particular representative examples of the present teachings.

In the description below, for purposes of explanation only, specific nomenclature is set forth to provide a thorough understanding of the present disclosure. However, it will be apparent to one skilled in the art that these specific details are not required to practice the teachings of the present disclosure.

Some portions of the detailed descriptions herein are presented in terms of algorithms and symbolic representations of operations on data bits within a computer memory. These algorithmic descriptions and representations are used by those skilled in the data processing arts to effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the below discussion, it is appreciated that throughout the description, discussions utilizing terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The algorithms presented herein are not inherently related to any particular computer or other apparatus. Various general-purpose systems, computer servers, or personal computers may be used with programs in accordance with the teachings herein, or it may prove convenient to construct a more specialized apparatus to perform the required method steps. The required structure for a variety of these systems will appear from the description below. It will be appreciated that a variety of programming languages may be used to implement the teachings of the disclosure as described herein.

Moreover, the various features of the representative examples and the dependent claims may be combined in ways that are not specifically and explicitly enumerated in order to provide additional useful embodiments of the present teachings. It is also expressly noted that all value ranges or indications of groups of entities disclose every possible intermediate value or intermediate entity for the purpose of an original disclosure, as well as for the purpose of restricting the claimed subject matter. It is also expressly noted that the dimensions and the shapes of the components shown in the figures are designed to help to understand how the present teachings are practiced, but not intended to limit the dimensions and the shapes shown in the examples.

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, well-known structures associated with servers, networks, displays, media handling, computers and/or processor/control systems have not been shown or described in detail to avoid unnecessarily obscuring descriptions of the embodiments.

Unless otherwise indicated, the functions described herein may be performed by executable code and instructions stored in a computer readable medium and running on one or more processor-based systems. However, state machines and/or hardwired electronic circuits may also be utilized. Further, with respect to the example processes described herein, not all of the process states need to be reached, nor do the states have to be performed in the illustrated order. Further, certain process states that are illustrated as being serially performed may be performed in parallel.

Similarly, unless expressly stated to be otherwise, while certain embodiments may refer to a Desktop Computer (“PC”) system or data device, other computer or electronic systems may be used as well, such as, without limitation, an interactive television, a network-enabled personal digital assistant (“PDA”), a network-enabled game console, a network-enabled entertainment device, a smart phone (e.g., with an operating system and on which a user may install applications) and the like.

In addition, while certain user inputs or gestures are described as being provided via phone key-presses, data entry via a keyboard, or by clicking a computer mouse or button, optionally, user inputs may be provided using other techniques, such as by voice or otherwise. The example screen layouts, appearance, and terminology as depicted and described herein, are intended to be illustrative and exemplary, and in no way limit the scope of the invention as claimed.

The terms, “for example,” “e.g.,” “in one/another aspect,” “in one/another scenario,” “in one/another version,” “in some configurations” “in some implementations,” “preferably,” “usually,” “typically,” “may,” and “optionally,” as used herein, are intended to be used to introduce non-limiting embodiments. Unless expressly stated otherwise, while certain references are made to certain example system components or services, other components and services may be used as well and/or the example components may be combined into fewer components and/or divided into further components. The terms, “member” and “user,” are used interchangeably. Members and users are subscribed to or enrolled in a network service or network of users.

The disclosed embodiments provide a secure centralized system and/or method to store the vital life information of the “system-involved” youth (i.e., youth who are connected to the child welfare system, juvenile justice system, public welfare system, foster care system, or combinations thereof) at by providing support to those “system-involved” youth in the areas of high school completion, employment, health care, postsecondary education, housing, and life-sustaining resources. Traditional document management systems do not provide the needed degrees of authorization and types of security features to different personnel (e.g., system administrators, case managers, general staff, youth, or the like) as do the disclosed embodiments of the system described herein. The disclosed system provides the requisite high level of security needed for the vital life documents of “system-involved” youth.

Generally, the different and various embodiments of the present disclosure cover a system and method to store and secure the life documents of the “system-involved” youth at care facilities in a protected, centralized system accessible via an interactive web application over Internet. In some embodiments, the interactive web application of the protected, centralized system is accessible from properly authenticated internet-capable devices. In other embodiments, the life documents of the “system-involved” youth are accessible via an interactive web site, but only from known, secure, physical locations to provide an additional layer of “in-person” authentication. As a result, at any given time, a “system-involved” youth with authorization from his/her case worker, can use the system to retrieve personal information in order to locate viable resources, continue education, find medical services, and the like. Since, “system-involved” youth are constantly shifting location, keeping their information in a stationary, electronically secured location can solve many of their prominent problems.

The “Life Document Electronic Security System For Youth” (LDESSY) described herein, offers tailored resources, ongoing educational services, and community referrals to the “system-involved” youth at a care facility via a web portal. Moreover, the LDESSY provides an individualized secure account for each case manager working on multiple “system-involved” youth. Each social worker can only access and edit the vital life documents of “system-involved” youth under his/her care by logging into his/her account at the LDESSY. This provides further security to the life documents of the “system-involved” youth by restricting the accessibility of the life documents only to the authorized personnel (e.g., the youth, his/her case worker and the system administrator) associated with each “system-involved” youth. Notably, in some embodiments, the “system-involved” youth do not have the editing rights to their vital life documents (since they are minors, and may sometimes have unstable backgrounds, appropriate supervision is provided). Only a social worker/case manager and the system administrator, with an individualized secure account at the LDESSY, can upload and edit the vital life document of a “system-involved” youth.

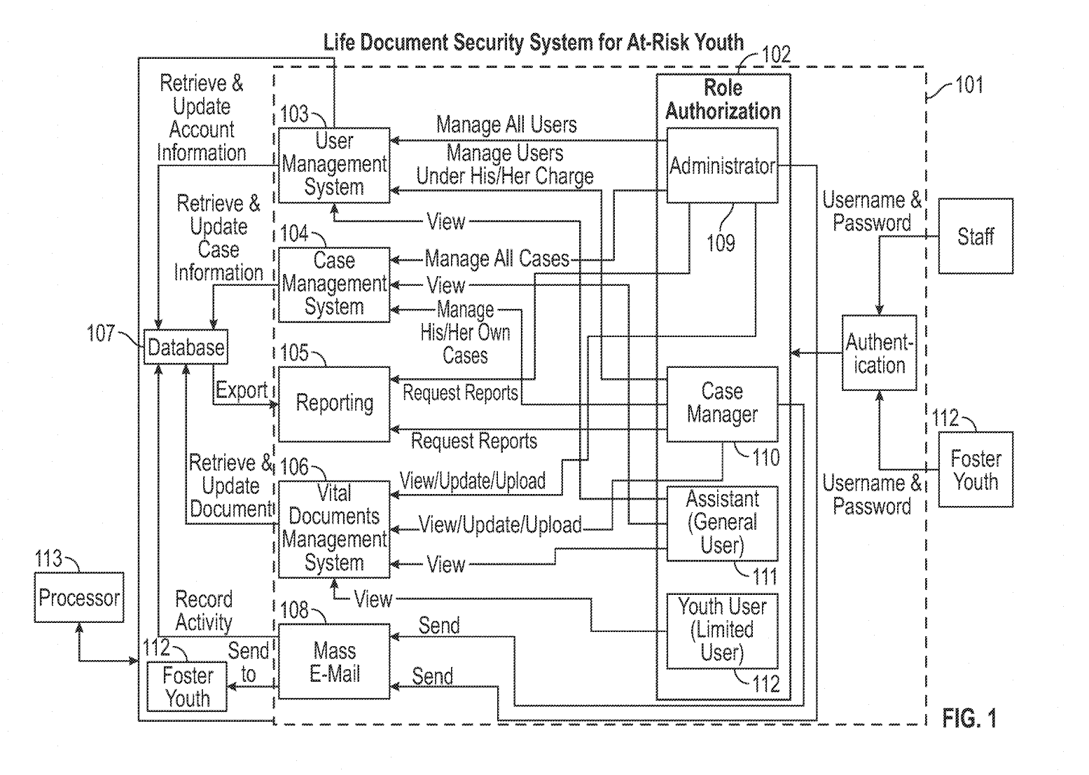

Referring now to the drawings, where like reference numerals denote like or corresponding parts throughout the drawings, and more particularly to FIG. 1, there is shown a relationship diagram between LDESSY and the different personnel (e.g., system administrator, social worker/case manager, “system-involved” youth, and the like) with authorization to access the LDESSY. The relationship diagram, as disclosed in FIG. 1, primarily comprises the LDESSY 101 section and a role authorization section 102, interconnected with each other via a plurality of functional elements. In one embodiment, the LDESSY 101 includes a user management system 103, a case management system 104, a reporting system 105, and a vital document management system 106, stored in a database 107. In some embodiments, the LDESSY 101 also includes a mass email system 108, the activity record of which is also stored in the database 107.

In one embodiment, the vital life documents (e.g., social security cards, birth certificates, photo identification, immunization records, training certificates, legal documents, school records, and the like) of the “system-involved” youth 112 are uploaded and stored in the database 107 of the LDESSY 101 by the designated case manager 110 of the youth 112. Notably, the Department of Children and Family Services authorizes the system to maintain the vital life documents described herein for “system-involved” youth under their care. Many of these life documents (e.g., social security card, birth certificate, driver's license) are illegal for most companies or individuals to maintain.

The role authorization section 102 of the relationship diagram of FIG. 1 includes one or more system administrators 109, one or more case managers 110, one or more assistants 111 to the case managers, and a plurality of “system-involved” youth 112. The system administrators 109 have the authorization to access, manage, and update/edit information regarding all the users of the LDESSY 101 stored in the database 107. The system administrator 109 accesses, manages, and updates/edits information regarding all the users of the LDESSY 101 using a processor 113 in communication with the database, and the user management system 103, the case management system 104, the vital document management system 106, and sections of the database 107. The system administrators 109 also have authorization to request demographic reports from the reporting system 105 and send group/mass emails to all the “system-involved” youth 112 users of the LDESSY 101.

The case management system 104 of the LDESSY 101 works as an applicant tracking system, electronic medical charting system, and a customer relationship management system. Specifically, the case management system 104 of the LDESSY 101 enables case managers 110 to electronically connect with clients, monitor service delivery, and record and report progress.

Each case manager of the one or more case managers 110 have the authorization to access, manage, and update/edit information regarding all of the “system-involved” youth 112 under his or her charge. The information regarding all of the “system-involved” youth 112 is stored in the database 107. The case manager 110 accesses, manages, and updates/edits information regarding all of the “system-involved” youth 112 under his or her charge in the LDESSY 101 using a processor 113 in communication with the database, and the user management system 103, the case management system 104, and the vital document management system 106 section of the database 107. This configuration is far more secure than a paper system in which a case manager could more easily access the information of other “system-involved” youth 112 not under his/her charge. The LDESSY 101 enables system administrators 109 to assign, reassign, and other change relationships between case manager 110 and “system-involved” youth 112 with the certainty that only the authorized personnel will have access to the information of the “system-involved” youth 112 in their charge. This security could not be achieved with paper systems where the access and return of paper files is simply too difficult to control.

In some embodiments, two different levels of authorized personnel are required to access vital life documents. For example, in some embodiments an individual youth and his/her case manager are both required to provide authentication in order for the individual youth to access his/her vital life documents. This provides another level of security to protect the vital life documents when it is determined to be necessary. In another aspect of some embodiments, authorized personnel are required to provide authentication from two different locations in order to access vital life documents. For example, in one such embodiment, an individual youth may provide authentication from a mobile device, but his/her case manager must also provide authentication from a “back end” location at a nonprofit organization in order for the individual youth to access his/her vital life documents. Again, this provides still another level of security to protect the vital life documents when it is determined to be necessary.

In another aspect of such an embodiment, the “system-involved” youth first contacts their case manager (by phone, in person, by email, text, or the like) to let them know that they are going on a job interview and will need access to their driver's license and social security number, both of which are stored within the database of the LDESSY. In some cases, “by phone” or “in person” contact is required so that the case manager can verify that it is their “system-involved” youth contacting them. In other embodiments, email, text, or other types of contact may be acceptable. Once the case manager has confirmed and authenticated the request from their “system-involved” youth, the “system-involved” youth can access their driver's license and social security number in response to the “system-involved” youth and the case manager both providing their proper ID/username and password.

In still another embodiment, the “system-involved” youth again first contacts their case manager (by phone, in person, or the like) to let them know that they are going on a job interview and will need access to their driver's license and social security number, both of which are stored within the database of the LDESSY. Again, the case manager verifies that it is their “system-involved” youth contacting them. Then in this embodiment (after the case manager provides his proper ID/username and password), the case manager moves the youth's driver's license and social security number from an inaccessible partition of the LDESSY database to a partition of the LDESSY database that is accessible by the “system-involved” youth after the “system-involved” youth provides their proper ID/username and password. The youth's driver's license and social security number only remain in the accessible partition of the LDESSY database (with the youth's proper ID/username and password) for a limited period of time. This embodiment provides an even greater level of security to the “system-involved” youth's information. Such an embodiment helps prevent a “system-involved” youth's information from being “hacked” or otherwise stolen if the “system-involved” youth's proper ID/username and password are compromised.

Each case manager 110 is also authorized to request demographic reports from the reporting system 105 and send emails to “system-involved” youth 112 users, of the LDESSY 101 either individually, categories, or in mass. Specifically, using the LDESSY, a case manager and a system administrator are authorized to generate a report of demographic and activity information of the “system-involved” youth and send mass emails to the “system-involved” youth users of the LDESSY. In some embodiments, the features of the LDESSY are customized to meet the individual need of a case manager and to further help the “system-involved” youth to achieve their full life potentials.

In one embodiment, the assistants 111 to the case managers only have the authorization to view the contents stored in the database 107 (and not the vital life documents). In another embodiment, the “system-involved” youth 112 who are registered users of the either individual, category, or in mass 101, have the authorization to view their own vital life documents from the database 107. In still another embodiment, the “system-involved” youth 112 who are registered users of the LDESSY 101 need the authorization of their case manager as well. In some embodiments, there may be another level of security access for an IT (Information Technology) worker that provides “code access” to the system but not “case access” to the LDESSY 101.

In order to access the LDESSY 101, every user (e.g., administrators 109, case managers 110, assistants 111 to the case managers, the “system-involved” youth 112, and the like) of the LDESSY 101 registers online to the LDESSY 101 by creating an online account. Once registered, a user can log into the LDESSY 101 by inputting his/her email address, password, a security code, and answer one or more personal security questions. In some embodiments, the security code is received at the phone number provided during the creation of the online account of the user. The verification code prevents login from a machine/robot. The personal security question provides a method for changing password. Mobile and email verification prevent login from unauthorized users. In some embodiments, the LDESSY 101 records whenever access is attempted and granted to the database 107. This feature helps ensure that case managers 110 (and others) are only accessing files in the database 107 of the LDESSY 101 for appropriate reasons since they know that there will be a record of their activity.

In some embodiments, a “system-involved” youth 112 cannot register directly online at the LDESSY 101. In order to access the LDESSY 101, a “system-involved” youth 112 first physically registers at the office (e.g., care facility) that administers and maintains the LDESSY 101. A social worker/case manager 110 assigned to the “system-involved” youth 112 then reviews the case and decides, based on the information presented by the “system-involved” youth 112 during the registration process, whether to accept their registration. If the case manager 110 is satisfied with the information presented by the LDESSY youth 112 during the registration, the case manager 110 accepts the case and logs into the LDESSY 101 to create a new case for the LDESSY youth 112. The case manager 110 then sends an email link to the LDESSY youth 112 to set up a security question and password for their personal account. Once the LDESSY youth 112 receives the email link from the case manager 110, they set up a security question and a password to login to the LDESSY 101. In some embodiments, the case manager 110 can also customize the personal account of the LDESSY youth 112 with different security settings depending on several factors, including the age and background of the LDESSY youth 112. For example, some LDESSY youth 112 who are registered users of the LDESSY 101 may have the authorization to view their own vital life documents from the database 107, while other LDESSY youth 112 who are registered users of the LDESSY 101 may need the authorization of their case manager as well.

In order to upload a life document in the database 107, the case manager 110 of the “system-involved” youth 112 under his/her charge logs into the LDESSY 101, and chooses the case of the “system-involved” youth 112 under their domain whose document they wish to upload. The case manager 110 then adds a document description, and requests the LDESSY 101 to upload the desired life document to the database 107. The LDESSY 101 processes the request by contacting the vital document management system 106 of the database 107 using the processor 113 in communication with the LDESSY 101. The vital document management system 106 receives the life document of the “system-involved” youth 112 and stores the document in the database 107 using the processor 113. Once the desired life document of the “system-involved” youth 112 is stored in the database 107, the “system-involved” youth 112 can access their vital life documents using the LDESSY 101 account. The case manager 110 can only access the vital life documents stored in the database 107 of the LDESSY youth 112 under their jurisdiction using their LDESSY 101 account.

Notably, a system administrator 109 can access the stored vital life documents in the database 107 of all the LDESSY youth 112 users of LDESSY 101. However, as described above, in some embodiments, the system records whenever access is attempted and/or granted to the database 107, as well as the changes that are made to the accounts. This feature helps ensure that system administrators 109 are only accessing files in the database 107 of the LDESSY 101 for appropriate reasons since they know that there will be a record of their activity.

In one embodiment, a case manager 110 or an administrator 109 of the LDESSY 101 can generate a report on a particular demographic data of the LDESSY youth 112 users of the LDESSY 101. In order to do so, the case manager 110 or the administrator logs in to the LDESSY 101. Once logged in, the case manager 110 or the administrator 109 chooses a report type based on the desired demographic data, adds a short description of the desired report, and requests the LDESSY 101 to generate a report on a particular demographic data of the registered LDESSY youth 112 of the LDESSY 101. The LDESSY 101 processes the request by contacting the reporting system 105 of the database 107 using the processor 113 in communication with the LDESSY 101. The reporting system 105 accesses the requested demographic data from the database 107 and creates the report on the desired demographic data of the “system-involved” youth 112 using the processor 113. Once the requested report is created, the case manager 110 or the administrator 109 can access the report via their LDESSY 101 account.

In one embodiment, a case manager 110 (or a system administrator 109) of the LDESSY 101 can send mass emails to all “system-involved” youth 112 users of the LDESSY 101. In order to do so, the case manager 110 (or the system administrator 109) logs into the LDESSY 101. Once logged in, the case manager 110 (or the system administrator 109) chooses a group of “system-involved” youth 112 based on the information the case manager 110 (or the system administrator 109) desires to share. Next, the case manager 110 (or the system administrator 109) adds a subject and content to the email, and requests the LDESSY 101 to send a mass email to the selected group of registered youth 112 of the LDESSY 101. Since the mass email may be sent to a selected group of registered youth 112, the mass email may still be a targeted email to the group of “system-involved” youth 112 selected by the case manager 110 (or the system administrator 109). The LDESSY 101 processes the request using the processor 113 in communication with the LDESSY 101 and sends the mass email to the desired group of LDESSY youth 112. The LDESSY 101 then stores a copy of the email in the database 107 using the processor 113.

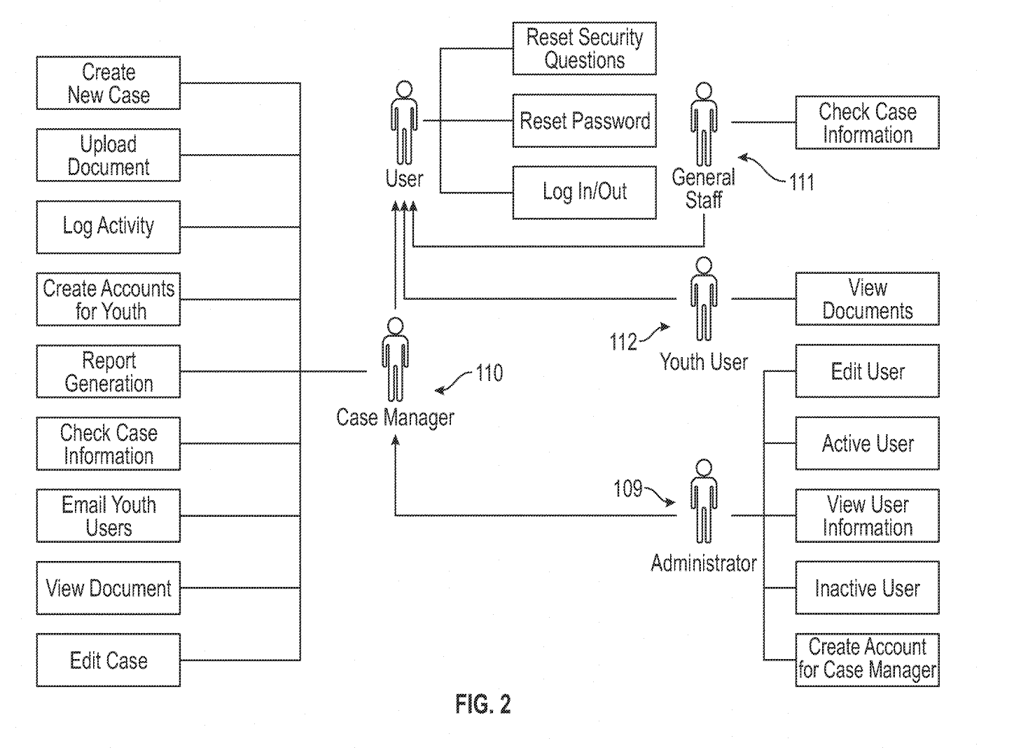

Referring now to FIG. 2, there is shown a diagram of different functions of different personnel (e.g., system administrators, case managers, “system-involved” youth, and the like), who are the users of the LDESSY 101, based on their authorization/security level. As shown in FIG. 2, the system administrators 109 have the authorization to view and edit information regarding all the users of the LDESSY 101, which are stored in the database 107. The system administrators 109 also have authorization to activate or inactivate any user's account. Additionally, in some embodiments, the system administrators 109 are authorized to create and edit a new case, as well as upload life documents of the “system-involved” youth 112. The system administrators 109 are also authorized to create accounts for case managers 110 and other users of the LDESSY 101.

As also shown in FIG. 2, a case manager 110 has the authorization to create a new account in the LDESSY 101 for the “system-involved” youth 112 under his or her supervision. Once the account is created in the LDESSY 101 for the “system-involved” youth 112, the case manager 110 creates a new case in the LDESSY 101 and uploads the vital life documents of the “system-involved” youth 112 to the LDESSY 101. The LDESSY 101 stores the life documents of the “system-involved” youth 112 to the database 107 for the case manager 110 or the you 112 to access later using their corresponding login ID and password. A case manager 110 also has the authorization to view the life documents, check the case information, and edit the case of the “system-involved” youth 112 under his or her supervision. In some embodiments, the case manager 110 generates demographic reports from the information of the “system-involved” youth 112 stored in the database 107 and sends mass emails to “system-involved” youth 112 users of the LDESSY 101. In some embodiments, the system administrators 109 and the case managers 110 log activity of the “system-involved” youth 112 in the LDESSY 101 and save the report of the activity for future use.

In one embodiment, the general staff working at the nonprofit organization (or maintenance center of the LDESSY 101) are authorized to check case information of the “system-involved” youth 112 stored in the database 107 of the LDESSY 101, but not access life documents from the LDESSY 101. As described above, in some embodiments, the “system-involved” youth 112 who are registered users of the LDESSY 101 have the authorization to view and download their own life documents from the LDESSY 101.

In one embodiment, access to the LDESSY 101 is provided to authorized users (e.g., administrators 109, case managers 110, general staff/assistants 111 to the case managers and the “system-involved” youth 112) of the LDESSY 101 only after they have registered online to the LDESSY 101 website by creating an online account. Once registered, an authorized user can log into the LDESSY 101 by inputting his/her email address, password, a security code, and answer to one or more personal security questions. Any user can also change the password and security question of their account.

In above disclosed embodiments, the system and method disclosed herein discusses securing vital life documents, such as, social security cards, birth certificates, picture identification, or the like, for the “system-involved” youth. In other embodiments, the disclosed system and method can be used to secure life documents for veterans or other adults who experience chronic homelessness, mental illness, physical/developmental disabilities, traumatic brain injuries, forms of dementia or memory loss, and/or low to no English literacy, which may prevent them from accurately identifying and securing their vital life documents. Moreover, the disclosed system and method may also be used for any other group or population who are unable to secure their vital life documents due to physical, psychological, medical, environmental, and/or circumstantial challenges.

Following are some examples of some support systems which can be used to implement the LDESSY 101. A person skilled in the art will appreciate that any other support systems can be used to implement the LDESSY 101 as well.

Sample System Requirements (e.g., these specifications and models are given as examples and not absolute requirements).

Back End:
Windows Server 2012+ and Linux 2.4+ operating systems in 32-bit or 64-bit modes.
MySQL Database
2 gigahertz (GHz) or faster 32-bit (×86) or 64-bit (×64) processor
4 gigabyte (GB) RAM (32-bit) or 8 GB RAM (64-bit)
32 GB available hard disk space (32-bit) or 64 GB (64-bit)
Broadband Internet Connection

End User:

Personal Computers:
a. Windows:
   i. General Requirements:
      1. Windows 7 or later
      2. 1 gigahertz (GHz) or faster 32-bit (×86) or 64-bit (×64) processor
      3. 1 gigabyte (GB) RAM (32-bit) or 2 GB RAM (64-bit)
      4. 16 GB available hard disk space (32-bit) or 20 GB (64-bit)
      5. Chrome, Firefox or Safari Browser
      6. Broadband Internet Connection
   ii. Supported Models:
      Any system Intel-based personal computer with 1 gigahertz (GHz) or faster 32-bit (×86) or 64-bit (×64) processor
b. Macintosh:
   i. General Requirements:
      1. OS X v10.6.8 or later
      2. 2 GB of memory
      3. 8 GB of available space
      4. Chrome, Firefox or Safari Browser
      5. Broadband Internet connection
   ii. Supported Models:
      1. iMac (Mid 2007 or newer)
      2. MacBook (late 2008 Aluminum, or early 2009 or newer)
      3. MacBook Pro (mid/late 2007 or newer)

Mobile Devices:
a. iOS (v7+)
   i. iPhone 5 or later
   ii. iPad 3 or later
b. Android (v4+)
   i. Samsung Galaxy S3 or later
c. Windows Phone (v8+)
   i. Nokia Lumia Icon or later

Various aspects of the systems, methods, functions, steps, features and the like corresponding thereto disclosed herein may be implemented on one or more computer systems using hardware, software, firmware, circuits, or combinations thereof. Hardware, software, firmware, and circuits respectively refer to any hardware, software, firmware, or circuit component. Computer systems referred to herein may refer to any computing device and vice versa (e.g., smart phone, mobile computing device, personal data assistant, tablet computer, laptop computer, desktop computer, other computing device, and the like). For example, each computer system or computing device in the systems described herein or any embodiment of a system disclosed herein may utilize one or more of the following components: a single-core or multi-core hardware processor (e.g., central processing unit or graphics processing unit) on which software instructions are executed (e.g., instructions corresponding to an operating system, an application program, an interpreter such as a virtual machine, or a compiler); a memory associated with and in connection with the hardware processor such as cache or other system memory that stores software instructions or other data that the hardware processor may access for processing; an input device (e.g., mouse, keyboard, touchscreen, and the like); an output device (e.g., display, touchscreen, printer, and the like); a network or communication interface that enables the computer system to communicate over a network or communication protocol; an application program having corresponding software instructions that are executable by a hardware processor. Connections between different computer systems and connections between different computer system components may be wired or wireless.

Virtualization computing techniques, cloud computing techniques, web application/website computing techniques, traditional and adaptive streaming techniques, and other computing techniques may be implemented by any embodiment of a system disclosed herein to enable and/or enhance the teachings described herein. For example, in a cloud computing embodiment, one or more servers (i.e., one or more computer systems) may store and execute software instructions corresponding to an application program based on input data received from client devices. In response to the input data received, the application program is executed accordingly, which results in graphical data being processed and output to the client devices for display on a display such as a touch screen on a smart phone or tablet computer.

As another example, in a web application or website embodiment, data representative of a user input may be transmitted to a server (i.e., a computer system) hosting the website for processing and storage in memory. In an application program embodiment, the application may be stored and executed locally on a user's computer system. In other embodiments, one or more components of the application program may be stored and executed on a server and the user's computer system. For example, a user may download the application program from an app store for an Android computing device, Blackberry computing device, Apple computing device, Windows computing device, Samsung computing device, other computing device, and the like. Execution of the application program on the user's computing device may require that the device transmit and receive data to and from one or more computing devices such as a server or other user's computing device. For example, an application may be downloaded from a server to a mobile device. Upon installation, the mobile device may communicate with a server.

One or more aspects of the systems disclosed herein may be located on (i.e., processed, stored, executed, or the like; or include one or more hardware or software components) a single computer system or may be distributed among a plurality of computer systems attached by one or more communication networks (e.g., internet, intranet, a telecommunications network, and the like). One or more components of a computer system may be distributed across one or more computer systems in communication with the computer system over a communication network. For example, in some embodiments, the systems disclosed herein may utilize one or more servers (i.e., one or more computer systems dedicated for a particular purpose in the system) that may be dedicated to serve the needs of one or more other computer systems or components across a communication network and/or system bus. The one or more servers may provide a central processing location for one or more aspects of the systems disclosed herein.

Again, various aspects of the systems, methods, function, and steps corresponding thereto disclosed herein may be implemented on one or more computer systems using hardware, software, firmware, or combinations thereof. Those of ordinary skill in the art will appreciate that one or more circuits and/or software may be used to implement the system and methods described herein. Circuits refer to any circuit, whether integrated or external to a processing unit such as a hardware processor. Software refers to code or instructions executable by a computing device using any hardware component such as a processor to achieve the desired result. This software may be stored locally on a processing unit or stored remotely and accessed over a communication network.

As disclosed herein, a processor or hardware processor may refer to any hardware processor or software processor. A software processor may include or otherwise constitute an interpreter that is executed by a corresponding hardware processor. A computer system according to any embodiment disclosed herein is configured to perform any of the described functions related to the various embodiments of the systems disclosed herein.

As disclosed herein, any method, function, step, feature, or result may be considered a module that may include software instructions that cause, when executed by a computing device, the desired method, function, step, feature, or result. Executed by a computing device includes execution by any hardware component (e.g., CPU, GPU, network interface, integrated circuits, other hardware components, and the like) of the computing device such as a hardware processor. Any module may be executed by a computing device (e.g., by a processor of the computing device). Any method, function, step, feature, result, and the like disclosed herein may be implemented by one or more software modules whether explicitly described or not. Individual components within a computing device may work together to accomplish a desired method, function, step, feature, or result. For example, a computing device may receive data and process the data. A simple example would be that a network interface receives the data and transmits the data over a bus to a processor.

Various aspects of the systems disclosed herein may be implemented as software executing in a computer system. The computer system may include a central processing unit (i.e., a hardware processor) connected to one or more memory devices, a graphical processing unit, input devices such as a mouse and keyboard, output devices such as speakers and a display, a network interface to connect to one or more other computer systems (e.g., one or more computer systems configured to provide a service such that function as a database), an operating system, a compiler, an interpreter (i.e., a virtual machine), and the like. The memory may be used to store executable programs and data during operation of the computer system. The executable programs may be written in a high-level computer programming language, such as Java or C++. Of course, other programming languages may be used since this disclosure is not limited to a specific programming language or computer system. Further, it is to be appreciated that the systems and methods disclosed herein are not limited to being executed on any particular computer system or group of computer systems.

Some methods, functions, steps, or features have been described as being executed by corresponding software by a processor. It is understood than any methods, functions, steps, features, or anything related to the systems disclosed herein may be implemented by hardware, software (e.g., firmware), or circuits despite certain methods, functions, steps, or features having been described herein with reference to software corresponding thereto that is executable by a processor to achieve the desired method, function, or step. It is understood that software instructions may reside on a non-transitory medium such as one or more memories accessible to one or more processors in the systems disclosed herein. For example, where a computing device receives data, it is understood that the computing device processes that data whether processing the data is affirmatively stated or not. Processing the data may include storing the received data, analyzing the received data, and/or processing the data to achieve the desired result, function, method, or step. It is further understood that input data from one computing device or system may be considered output data from another computing device or system, and vice versa. It is yet further understood that any methods, functions, steps, features, results, or anything related to the systems disclosed herein may be represented by data that may be stored on one or more memories, processed by one or more computing devices, received by one or more computing devices, transmitted by one or more computing devices, and the like.

The various embodiments and examples described herein are provided by way of illustration only and should not be construed to limit the claimed invention, nor the scope of the various embodiments and examples. Those skilled in the art will readily recognize various modifications and changes that may be made to the claimed invention without following the example embodiments and applications illustrated and described herein, and without departing from the true spirit and scope of the claimed invention, which is set forth in the following claims. In addition, various embodiments may be combined. Therefore, reference to an embodiment, one embodiment, in some embodiments, in other embodiments, and the like does not preclude one or more methods, functions, steps, features, results, hardware implementations, or software implementations of different embodiments from being combined. Further, reference to an embodiment, one embodiment, in some embodiments, in other embodiments, examples, and the like provides various aspects that may or may not be combined with those of one or more different embodiments and/or examples.

1. A protection system for electronically securing and enabling authorized access to one or more life documents of at-risk system-involved youth, the system comprising:
a server including a database that stores life documents in accounts associated with a plurality of youth users of the system, the database further including a user management system that controls authorization and security levels for system administrator users, case manager users, and youth users, wherein system administrator users have full access and editing privileges to all the accounts associated with the plurality of youth users, case manager users have full access and editing privileges to only accounts associated with youth users to which they have been assigned, and youth users only have access privileges to their own accounts;
a server processor in communication with the database, wherein the processor is configured to execute instructions to access or edit accounts of the plurality of youth users depending on the authorization and security level of a requesting user, wherein the editing of an account of a youth user includes uploading a life document to an account, updating information associated with a youth user, or combinations thereof; and
a network that connects the server to end user terminals associated with the system administrator users and case manager users, wherein the network is configured to receive and process requests from youth users;
wherein system administrator users are authorized to change assignments between case manager users and youth users, which immediately alters youth users accounts to which the case manager users have access and editing privileges, terminating access to prior assigned youth users accounts and granting access to newly assigned youth users accounts.

2.-20. (canceled)
